(19) United States
(12) Patent Application Publication
(10) Pub. No.: US 2025/0261879 A1
(43) Pub. Date: Aug. 21, 2025

(54) METHOD FOR GAIT INFORMATION PROCESSING

(71) Applicant: Tianjin University, Tianjin (CN)

(72) Inventors: Lin MENG, Tianjin (CN); Dong MING, Tianjin (CN); Xinge LI, Tianjin (CN); Zhimin ZHENG, Tianjin (CN); Jun PANG, Tianjin (CN); Rui XU, Tianjin (CN)

(73) Assignee: Tianjin University, Tianjin (CN)

(21) Appl. No.: 19/026,432

(22) Filed: Jan. 17, 2025

Related U.S. Application Data
(63) Continuation of application No. PCT/CN2024/139939, filed on Dec. 17, 2024.

(30) Foreign Application Priority Data
Feb. 19, 2024 (CN) 202410181756.4

(51) Int. Cl. A61B 5/11

(52) U.S. Cl. CPC A61B 5/112

(57) ABSTRACT

A method for processing gait information can be applied in inertial sensing and intelligent medical technology for Parkinson's disease. The method includes: detecting the motion state of a target object in response to receiving confirmation information indicating that the target object has taken the target medication; when the target object is walking, collecting gait information during straight-line walking; in response to detecting that the target object transitions from straight-line walking to another type of gait, and the number of gait information collections exceeds a predetermined threshold, processing the collected gait information with a target function to obtain gait parameters; and finally sending the gait parameters to a mobile communication terminal for further abnormality detection.

### CROSS REFERENCE TO THE RELATED APPLICATIONS

This application is the continuation application of International Application No. PCT/CN2024/139939, filed on Dec. 17, 2024, which is based upon and claims priority to Chinese Patent Application No. 202410181756.4, filed on Feb. 19, 2024, the entire contents of which are incorporated herein by reference.

### TECHNOLOGY FIELD

The present invention relates to the field of inertial sensing technology and intelligent medical technology for Parkinson's disease, particularly to a method for gait information processing.

### BACKGROUND

Parkinson's disease (PD) is the second most common chronic neurodegenerative disease after Alzheimer's disease, characterized primarily by bradykinesia, tremors, rigidity, and gait abnormalities. With the development of medical technology, electronic devices can be used to collect gait information from patients with Parkinson's disease, providing technical support for medical care.

During the implementation of the concept of this invention, the inventors discovered that the collection of a large amount of invalid information leads to high resource consumption by electronic devices.

### SUMMARY

In view of the above problems, the present invention provides a method for gait information processing.

According to a first aspect of the present invention, a gait information processing method is provided, which is applicable to wearable devices, comprising: In response to receiving confirmation information that characterizes the target object confirming the use of a specific medication, detecting the movement state of the target object; When the movement state of the target object is walking, collecting straight-line walking information of the target object; In response to detecting that the target object has transitioned from straight-line walking to other types of walking, if the number of times straight-line walking information is collected is greater than or equal to a predetermined collection threshold, processing the straight-line walking information of the target object using a target function to obtain gait parameters; Transmitting the gait parameters to a mobile communication terminal for the mobile communication terminal to perform anomaly detection on the gait parameters.

According to one embodiment of the present invention, the walking information includes a first segment of walking information, a second segment of walking information, and a third segment of walking information collected sequentially in chronological order. The gait information processing method further comprises: Determining the first straight-line walking condition information of the target object based on the first segment and the third segment of walking information; In response to the first straight-line walking condition information indicating that the target object transitions from straight-line walking to turning, determining the second straight-line walking condition information of the target object based on the second segment of walking information; Determining that the target object has transitioned from straight-line walking to other types of walking based on the second straight-line walking condition information.

According to another embodiment of the present invention, the first segment of walking information includes first walking position coordinates, and the third segment of walking information includes second walking position coordinates. Determining the first straight-line walking condition information of the target object includes: inputting the first walking position coordinates into a fitting line function to obtain a first fitted line, where the first fitted line includes the first predicted position coordinates corresponding to the first walking position coordinates, and the sum of squares of the error between the first predicted position coordinates and the first walking position coordinates is minimized; inputting the second walking position coordinates into the fitting line function to obtain a second fitted line, where the second fitted line includes the second predicted position coordinates corresponding to the second walking position coordinates, and the sum of squares of the error between the second predicted position coordinates and the second walking position coordinates is minimized. When the first error sum of squares and the second error sum of squares meet the predetermined error condition, determining the first straight-line walking condition information of the target object based on the slope of the first fitted line and the slope of the second fitted line.

According to another embodiment of the present invention, the second segment of walking information includes Q yaw angle values, which are arranged in the walking sequence of the target object, where Q is a positive integer greater than 1. In response to the first straight-line walking condition information indicating that the target object transitions from straight-line walking to turning, determining the second straight-line walking condition information of the target object based on the second segment of walking information, comprising: determining the difference between the qₜₕ yaw angle value and the (q+1)ₜₕ yaw angle value among the Q yaw angle values to obtain Q−1 differences, where q is a positive integer less than Q; in response to the sum of the Q−1 differences being greater than or equal to a predetermined difference threshold, generating information indicating the turning of the target object; in response to the sum of the Q−1 differences being less than the predetermined difference threshold, generating information indicating the continued straight-line walking of the target object.

According to another embodiment of the present invention, the gait parameters include swing phase duration, stance phase duration, foot height, stride width, stride length, pitch angle velocity variation coefficient during the landing phase, pitch angle velocity variation coefficient during the push-off phase, foot height variation coefficient, stride width variation coefficient, and stride length variation coefficient.

According to a second aspect of the present invention, a gait information processing method is provided, applicable to a mobile communication terminal, comprising: Receiving gait parameters from a wearable device, wherein the gait parameters are obtained when the wearable device detects that the target object has transitioned from straight-line walking to other types of walking, and the number of collections of straight-line walking information of the target object is greater than or equal to a predetermined collection threshold, and the gait information is processed using a target function; Performing anomaly detection on the gait parameters to obtain an anomaly detection result.

According to another embodiment of the present invention, performing anomaly detection on the gait parameters to obtain an anomaly detection result includes: in response to the number of days of collected walking information being equal to a predetermined day threshold, using a first gait parameter anomaly detection algorithm to process the gait parameters to obtain the first anomaly detection result corresponding to the change pattern of the gait parameters; using a second gait parameter anomaly detection algorithm to process the gait parameters to obtain a second anomaly detection result corresponding to the deviation of the gait parameters.

According to an embodiment of the present invention, there are M sets of gait parameters, where each set corresponds to one of M medication periods within a single day. These M sets of gait parameters are arranged according to the walking time, and M is a positive integer greater than 1. The first anomaly detection result is obtained by processing the gait parameters using the first gait parameter anomaly detection algorithm, which corresponds to the change pattern of the gait parameters. This includes: calculating the first-order forward difference value between the (m−1)ₜₕ set and the mₜₕ set of gait parameters, where m is a positive integer greater than 1 and less than or equal to M; calculating the second-order forward difference value between the (m−2)ₜₕ and (m−1)ₜₕ sets of first-order forward difference values from the M−1 sets of first-order forward difference values; generating the first anomaly detection result indicating no abnormal condition in the gait parameters in response to the second-order forward difference values being greater than a predetermined threshold; generating the first anomaly detection result indicating the presence of an abnormal condition in the gait parameters in response to the second-order forward difference values being less than or equal to the predetermined threshold.

According to an embodiment of the present invention, the mₜₕ set of gait parameters includes K gait parameters corresponding to K detection periods. The second anomaly detection result is obtained by processing the gait parameters with the second gait parameter anomaly detection algorithm. The second anomaly detection result includes: determining the statistical value of the K gait parameters; determining the K differences between the statistical value and the K gait parameters; generating the second anomaly detection result indicating no abnormal condition in the gait parameters if all K differences are less than or equal to a predetermined threshold; generating the second anomaly detection result indicating the presence of an abnormal condition in the gait parameters if any of the K differences is greater than the predetermined threshold.

According to an embodiment of the present invention, if both the first and second anomaly detection results indicate that no anomalies are present in the gait parameters, the gait parameters are sent to a server for storage. If at least one of the first and second anomaly detection results indicates the presence of anomalies, an information retrieval request is sent to the wearable device to obtain the walking information corresponding to the gait parameters with abnormalities stored in the device's cache. This walking information is then sent to the server for storage, alongside the gait parameters corresponding to the abnormal conditions.

The gait information processing method provided by the present invention adapts the next operation based on confirmation information, the target object's motion state, walking condition, the number of times walking information is collected, and the daily collection cycle count. This avoids wasting power resources of wearable devices and prolongs their standby time. Moreover, by avoiding unnecessary operations, the computational power of wearable devices is fully utilized to process only the necessary walking information, reducing overall resource consumption and preventing the processing of invalid walking information.

Furthermore, since only gait parameters are transmitted to the mobile communication terminal, the amount of information sent to the mobile communication terminal is reduced, thereby improving the information processing efficiency of the mobile communication terminal and conserving its resources.

### BRIEF DESCRIPTION OF THE DRAWINGS

The above description of the present invention, as well as other objectives, features, and advantages, will become clearer with reference to the following accompanying drawings.

FIG. 1 shows an application scenario of the gait information processing method according to an embodiment of the present invention.

FIG. 2 shows a flowchart of the gait information processing method according to an embodiment of the present invention.

FIG. 3 shows a schematic view of the portable insole according to an embodiment of the present invention.

FIG. 4 shows a state monitoring framework based on the SVM model according to an embodiment of the present invention.

FIG. 5 shows a schematic view of the straight-line and turning trajectories according to an embodiment of the present invention.

FIG. 6 shows a schematic of turn detection according to an embodiment of the present invention.

FIG. 7 shows a flowchart for generating the second straight-line walking condition information according to an embodiment of the present invention.

FIG. 8 shows another flowchart of the gait information processing method according to an embodiment of the present invention.

FIG. 9 shows a schematic view of the normal changes in gait parameters after medication according to an embodiment of the present invention.

FIG. 10 shows a schematic view of another embodiment of the gait information processing method according to the present invention.

FIG. 11 shows a structural block diagram of the wearable device according to an embodiment of the present invention.

FIG. 12 shows a structural block diagram of the mobile communication terminal according to an embodiment of the present invention.

FIG. 13 shows a block diagram of the electronic equipment suitable for implementing the gait information processing method according to an embodiment of the present invention.

### DETAILED DESCRIPTION OF THE EMBODIMENTS

The following detailed description is provided in connection with the accompanying drawings to further illustrate the embodiments of the present invention. However, it should be understood that these descriptions are merely exemplary and are not intended to limit the scope of the invention. Many specific details are provided in the following detailed description for a thorough understanding of the embodiments of the present invention. However, one or more embodiments can clearly be implemented without these specific details. Furthermore, to avoid unnecessarily confusing the essence of the present invention, descriptions of well-known structures and techniques have been omitted.

The terms used herein are intended to describe particular embodiments only and are not intended to limit the invention. The terms “comprise” and “include” indicate the presence of specified features, steps, operations, or components but do not preclude the presence or addition of one or more other features, steps, operations, or components.

All terms (including technical and scientific terms) used herein should be understood by those skilled in the art in accordance with their commonly understood meanings unless otherwise defined. Note that the terms used herein should be interpreted to have a meaning consistent with the context of the specification, and not in an idealized or overly rigid manner.

When using expressions like “at least one of A, B, and C” in this specification, it should be interpreted as generally understood by those skilled in the art (e.g., “a system having at least one of A, B, and C” includes but is not limited to a system having A, or B, or C, or A and B, or A and C, or B and C, or A, B, and C).

In the technical solution of the present invention, user information (including but not limited to personal information, user image information, and user equipment information, such as location information) and data (including but not limited to data used for analysis, stored data, and displayed data) are authorized by users or fully authorized by all parties. The collection, storage, use, processing, transmission, provision, disclosure, and application of relevant data comply with relevant national and regional laws, regulations, and standards. Necessary confidentiality measures are taken without violating public order and good customs, and appropriate operational options are provided to users for authorization or refusal.

According to an embodiment of the present invention, the health management system for Parkinson's disease may include a mobile communication terminal, a wearable device, and a server.

The inventors discovered that elderly patients with neurodegenerative diseases find it difficult to flexibly use applications on mobile communication terminals to assist medical staff in collecting disease information. Therefore, the collected information often fails to meet the needs of medical staff. Neurodegenerative diseases may include Parkinson's disease and others. For example, in medication management, it is necessary to collect gait information from patients after multiple exercises. Since medical staff need to analyze the changing trends of gait parameters over time, the quality of the collected gait information is highly demanding. Parkinson's disease can be divided into “on-phase” and “off-phase.” Based on these phases, information such as gait conditions during the effective period of medication can be evaluated. Medical staff need to analyze both the daily information collected and the trends related to medication dates.

For wearable devices, such as health smartwatches, the data collection mode needs to be manually adjusted by the user. Since the collection mode is a fixed paradigm set at the time of manufacture, health smartwatches struggle to automatically identify which data is valid and which should be uploaded based on the disease's needs and the required quality of motion data. Moreover, for home management of Parkinson's disease, wearable devices need to remain in standby mode for long periods and periodically upload the collected information. Due to this, the information uploaded may include invalid or unusable data, consuming the wearable device's limited battery and communication bandwidth. For data sent to a mobile communication terminal, invalid or unusable information wastes storage space and introduces significant noise to manual analysis by doctors, leading to resource wastage.

In view of this, embodiments of the present invention provide a gait information processing method applied to wearable devices, comprising: in response to receiving confirmation information indicating that the target object has used the specified medication, detecting the movement state of the target object. When the target object is in a walking state, collecting the straight-line walking information of the target object. In response to detecting that the target object transitions from straight-line walking to another type of walking, if the collection count of the straight-line walking information is greater than or equal to a predetermined threshold, and the daily collection cycle of the straight-line walking information is greater than or equal to a predetermined collection cycle threshold, processing the straight-line walking information of the target object using a target function to obtain gait parameters. These gait parameters are then sent to the mobile communication terminal for anomaly detection.

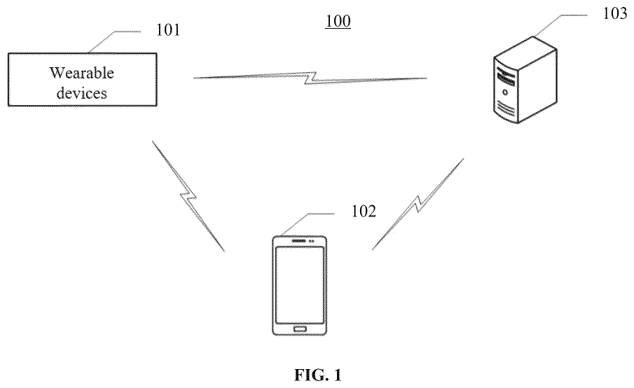

FIG. 1 shows an application scenario diagram of the gait information processing method according to an embodiment of the present invention.

As shown in FIG. 1, the application scenario 100 according to this embodiment includes a wearable device 101, a mobile communication terminal 102, and a server 103. The network provides a communication link medium between the wearable device 101, the mobile communication terminal 102, and the server 103. The network can include various types of connections, such as wired or wireless communication links, or optical fiber cables, etc.

The user can interact with the server 103 through the mobile communication terminal 102 via the network to receive or send messages, etc. The mobile communication terminal 102 may have various communication client applications installed, such as shopping apps, web browsers, search apps, instant messaging tools, email clients, social media platforms, etc. (These are just examples).

The wearable device can be an insole-type device based on a foot arch position 6-axis sensor, such as a foot IMU (Inertial Measurement Unit). Among the 6-axis sensors, 3 axes are used for measuring acceleration, and the other 3 axes are used for measuring angular velocity.

The mobile communication terminal 102 can be any electronic device with a display screen that supports web browsing, including but not limited to smartphones, tablets, laptops, and desktop computers, etc.

The server 103 can be a server that provides various services, such as a backend management server supporting websites browsed by the user using the mobile communication terminal 102 (just an example). The backend management server can analyze the received user requests and data, process them, and then send the results (such as webpages, information, or data generated or retrieved based on the user's requests) back to the mobile communication terminal.

It should be noted that the gait information processing method provided by the embodiments of the present invention can generally be executed by the wearable device 101 or the mobile communication terminal 102. It should be understood that the number of wearable devices 101, mobile communication terminals 102, and servers 103 shown in FIG. 1 is merely for illustration. Based on implementation requirements, there can be any number of wearable devices 101, mobile communication terminals 102, and servers 103.

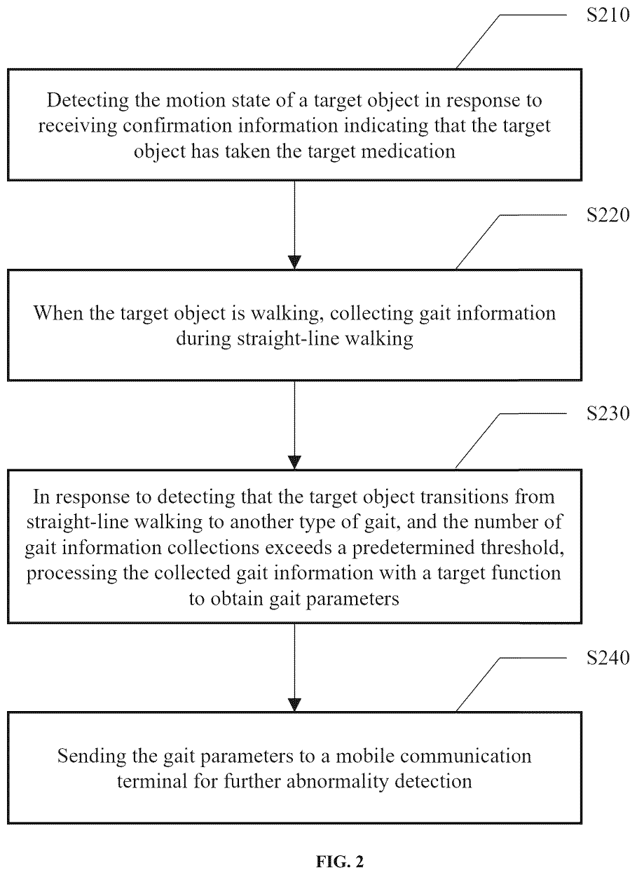

The gait information processing method of the embodiment of the invention will be described in detail through FIGS. 2 to 10, based on the application scenario described in FIG. 1.

FIG. 2 shows a flowchart of the gait information processing method according to an embodiment of the present invention.

As shown in FIG. 2, the gait information processing method of this embodiment can be applied to a wearable device. The method includes steps S210 to S240.

In step S210, in response to receiving confirmation information indicating that the target object has used the specified medication, the motion state of the target object is detected.

In step S220, when the motion state of the target object is walking, the straight-line walking information of the target object is collected.

In step S230, in response to detecting that the target object has transitioned from straight-line walking to another type of walking, and if the number of times the straight-line walking information is collected is greater than or equal to a predetermined collection count threshold, and the number of daily collection cycles of the straight-line walking information is greater than or equal to a predetermined collection cycle threshold, the straight-line walking information is processed using a target function to obtain gait parameters.

In step S240, the gait parameters are sent to the mobile communication terminal for anomaly detection of the gait parameters.

According to the embodiment of the present invention, the target object can be the object from which walking information is collected using a wearable device. The target object may be a person suffering from Parkinson's disease or other conditions. The medication that the target object uses can be targeted at conditions such as Parkinson's disease.

According to the embodiment of the present invention, the motion state of the target object can be used to characterize the activity the target object is performing. The motion state can include sitting, lying, or walking, etc.

According to the embodiment of the present invention, walking information can include the acceleration and angular velocity collected by the IMU during walking, but is not limited to this. Walking information can also include the position of the target object's foot, as well as the moments when different parts of the foot contact the ground.

According to the embodiment of the present invention, during a single walking detection, the target object may take a total of T steps, where I is a positive integer greater than 3.

According to the embodiment of the present invention, the gait parameters include swing phase duration, stance phase duration, foot height, stride width, stride length, pitch angle velocity variation coefficient during foot landing, pitch angle velocity variation coefficient during foot push-off, stride height variation coefficient, stride width variation coefficient, and stride length variation coefficient.

The target function may include functions for calculating the duration of the swing phase for one foot, the duration of the stance phase for one foot, foot height calculation function, stride width calculation function, stride length calculation function, pitch angle velocity variation coefficient during foot landing calculation function, pitch angle velocity variation coefficient during foot push-off calculation function, stride height variation coefficient calculation function, stride width variation coefficient calculation function, and stride length variation coefficient calculation function.

According to the embodiment of the present invention, the swing phase duration calculation function for one foot can be as follows:

$${swD}_{i} = {T}_{i + 1}^{HS} - {T}_{i}^{0} \qquad ( 1 )$$

Where swDᵢ represents the swing phase duration for one foot. Tᵢ⁰ represents the moment when the toe of the i^{th} step in the walking information lifts off the ground. Tᵢ₊₁^{HS} represents the moment when the heel of the (i+1)^{th} step in the walking information makes contact with the ground.

According to an embodiment of the present invention, the stance phase duration calculation function for one foot can be expressed as follows:

$$s t {D}_{i} = {T}_{i}^{0} - {T}_{i}^{HS} \qquad ( 2 )$$

where stDᵢ represents the stance phase duration for one foot. Tᵢ^{HS} represents the moment when the heel of the i^{th} step in the walking information makes contact with the ground. Tᵢ⁰ represents the moment when the toe of the i^{th} step in the walking information lifts off the ground.

According to an embodiment of the present invention, the foot height calculation function for one foot can be expressed as follows:

$$s {H}_{i} = {s}_{z , i}^{max} - {s}_{z , i}^{min} \qquad ( 3 )$$

where sHᵢ represents the foot height for one foot. s_{z,i}^{max} represents the highest vertical position of the foot during the i^{th} step in the walking information. s_{z,i}^{min} represents the lowest vertical position of the foot during the i^{th} step in the walking information.

According to an embodiment of the present invention, the stride width calculation function for one foot can be expressed as follows:

$$s {W}_{i} = {s}_{y , i}^{max} - {s}_{y , i}^{min} \qquad ( 4 )$$

where sWᵢ represents the stride width for one foot. s_{y,i}^{max} represents the furthest lateral position of the foot during the i^{th} step in the walking information. s_{y,i}^{min} represents the closest lateral position of the foot during the i^{th} step in the walking information.

According to an embodiment of the present invention, the stride length calculation function for one foot can be expressed as follows:

$$s {L}_{i} = {s}_{x , i}^{max} - {s}_{x , i}^{min} \qquad ( 5 )$$

where sLᵢ represents the stride length for one foot. s_{x,i}^{max} represents the furthest longitudinal (forward/backward) position of the foot during the i^{th} step in the walking information. s_{x,i}^{min} represents the closest longitudinal position of the foot during the i^{th} step in the walking information.

According to an embodiment of the present invention, the pitch angle velocity variation coefficient calculation function during foot landing can be expressed as follows:

$${hsPv}_{CV} = \frac{σ ( hsPv )}{μ ( hsPv )} \qquad ( 6 )$$

$$σ ( hsPv ) = \sqrt{\frac{{\sum }_{i = 1}^{Count} {( {hsPv}_{i} - μ ( hsPv ) )}^{2}}{Count}} \qquad ( 7 )$$

$$μ ( hsPv ) = \frac{{\sum }_{i = 1}^{Count} {hsPv}_{i}}{Count} \qquad ( 8 )$$

where hsPv_{CV} represents the pitch angle velocity variation coefficient during foot landing for one foot. hsPv_{CV} represents the pitch angle velocity of the foot during the i^{th} gait cycle's loading phase (LP) in the walking information. Count represents the total number of gait cycles in the walking information. μ(hsPv) represents the mean of the pitch angle velocity during foot landing for one foot. σ(hsPv) represents the standard deviation of the pitch angle velocity during foot landing for one foot.

According to an embodiment of the present invention, the pitch angle velocity variation coefficient calculation function during the foot push-off phase for one foot can be expressed as follows:

$${toPv}_{CV} = \frac{σ ( toPv )}{μ ( toPv )} \qquad ( 9 )$$

$$σ ( toPv ) = \sqrt{\frac{{\sum }_{i = 1}^{Count} {( {toPv}_{i} - μ ( toPv ) )}^{2}}{Count}} \qquad ( 10 )$$

$$μ ( hsPv ) = \frac{{\sum }_{i = 1}^{Count} {hsPv}_{i}}{Count} \qquad ( 11 )$$

where toPv_{CV} represents the pitch angle velocity variation coefficient during the foot push-off phase for one foot. toPv_{CV} represents the pitch angle velocity of the foot during the i^{th} gait cycle's push-off phase in the walking information. μ(hsPv) represents the mean of the pitch angle velocity during foot push-off for one foot. σ(toPv) represents the standard deviation of the pitch angle velocity during foot push-off for one foot. Count represents the total number of gait cycles in the walking information.

According to an embodiment of the present invention, the stride height variation coefficient calculation function for one foot can be expressed as follows:

$$s {H}_{CV} = \frac{σ ( sH )}{μ ( sH )} \qquad ( 12 )$$

$$σ ( sH ) = \sqrt{\frac{{\sum }_{i = 1}^{Count} {( {sH}_{i} - μ ( sH ) )}^{2}}{Count - 1}} \qquad ( 13 )$$

$$μ ( sH ) = \frac{{\sum }_{i = 1}^{Count} {sH}_{i}}{Count} \qquad ( 14 )$$

where sH_{CV} represents the stride height variation coefficient for one foot. σ(sH) represents the standard deviation of the stride height for one foot. μ(sH) is the average stride height for one foot. sHᵢ represents the foot height of the i^{th} step in the walking information. Count represents the total number of steps in the walking information.

According to an embodiment of the present invention, the stride width variation coefficient calculation function for one foot can be expressed as follows:

$$s {W}_{C V} = \frac{σ ( sW )}{μ ( sW )} \qquad ( 15 )$$

$$σ ( sW ) = \sqrt{\frac{{\sum }_{i = 1}^{Count} {( {sW}_{i} - μ ( sW ) )}^{2}}{Count - 1}} \qquad ( 16 )$$

$$μ ( sW ) = \frac{{\sum }_{i = 1}^{Count} {sW}_{i}}{Count} \qquad ( 17 )$$

where sW_{CV} represents the stride width variation coefficient for one foot. σ(sW) represents the standard deviation of the stride width for one foot. μ(sW) represents the average stride width for one foot. sWᵢ represents the stride width of the i^{th} step in the walking information. Count represents the total number of steps in the walking information.

According to an embodiment of the present invention, the stride length variation coefficient calculation function for one foot can be expressed as follows:

$${sL}_{CV} = \frac{σ ( sL )}{μ ( sL )} \qquad ( 18 )$$

$$σ ( sL ) = \sqrt{\frac{{\sum }_{i = 1}^{Count} {( {sL}_{i} - μ ( sL ) )}^{2}}{Count - 1}} \qquad ( 19 )$$

$$μ ( sL ) = \frac{{\sum }_{i = 1}^{Count} {sL}_{i}}{Count} \qquad ( 20 )$$

where sL_{CV} represents the stride length variation coefficient for one foot. sLᵢ represents the stride length of the i^{th} step in the walking information. σ(sL) represents the standard deviation of the stride length for one foot. μ(sL) is the average stride length for one foot. Count represents the total number of walking steps in the walking information.

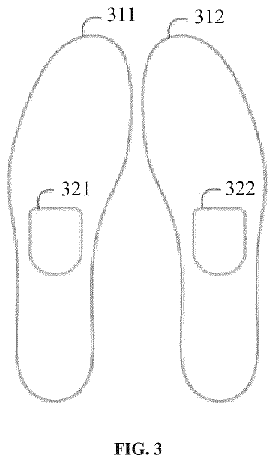

FIG. 3 shows a schematic view of a portable insole according to an embodiment of the present invention.

The wearable device in the form of insoles worn by the target object, as shown in FIG. 3, includes a first micro-sensor 321 and a second micro-sensor 322 based on a foot arch position 6-axis sensor for foot IMU (Inertial Measurement Unit). These sensors can be embedded in the first insole 311 and the second insole 312 to avoid interfering with the target object's daily movements and to allow for long-term wear. The first micro-sensor 321 for the left foot is embedded in the first insole 311, while the second micro-sensor 322 for the right foot is embedded in the second insole 312.

The acquisition module in the insoles can be used to collect three-axis acceleration and three-axis angular velocity walking data output by the foot IMU in real-time.

The preprocessing module in the insoles can be used to handle missing values, detect anomalies, and denoise the collected three-axis acceleration and angular velocity data. The processed acceleration and angular velocity data are then obtained.

The quality discrimination module in the insoles can be used to determine in real-time whether the target object's motion state is walking. It ensures that walking data is only cached and gait parameters are calculated when the target object is walking, improving the efficiency of wearable device storage and computational resources.

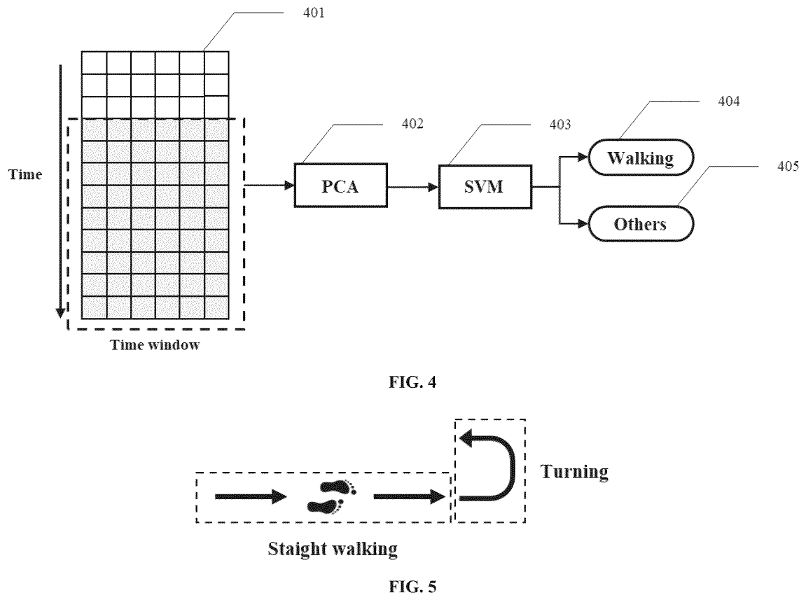

FIG. 4 shows a schematic diagram of the state monitoring framework based on an SVM model according to an embodiment of the present invention.

The quality discrimination module can also process the pre-processed acceleration and angular velocity data through a trained SVM (Support Vector Machines) algorithm 403, as shown in FIG. 4, to determine whether the target object is currently walking. PCA (Principal Component Analysis) algorithm 402 can be used to process the six-axis data 401, which is the pre-processed acceleration and angular velocity data collected by the six-axis sensor. The reduced feature information is then processed by the trained SVM 403 to determine the current motion state of the target object. The target object's current motion state may include walking type 404 or other types 405.

FIG. 5 shows a schematic view of the straight-line and turning trajectories according to an embodiment of the present invention.

According to the embodiment of the present invention, the target object may exhibit the motion pattern shown in FIG. 5 during free walking. This motion pattern consists of two parts: straight walking and turning. Since the most crucial part for characterizing the gait features of the target object is the straight walking information, the walking information collection paradigm involves both straight walking and turning. The turning gait action can be used as a marker to indicate the end of the walking information collection.

As shown in FIGS. 4 and 5, the PCA algorithm 402 and SVM algorithm 403 can be used to determine whether the target object's current state is straight walking or turning. The current state will only be determined as straight walking or turning using the PCA algorithm 402 and SVM algorithm 403 if the number of straight walking steps for the target object reaches a predetermined collection threshold.

According to an embodiment of the present invention, if the motion state of the target object is detected to be non-walking, a prompt command can be sent to the mobile communication terminal to remind the target object to perform a gait detection.

When the motion state of the target object is detected to be walking, the collected walking information can be stored, and a peak detection algorithm can be used to process the collected walking information to calculate the number of collections.

According to an embodiment of the present invention, the target object needs to take medication multiple times a day. For each medication, the target object needs to undergo multiple walking detections. During each walking detection, the walking information needs to be collected multiple times within the detection cycle. The daily collection cycle count refers to the number of walking detections performed after the target object takes medication in a single day. The predetermined collection cycle threshold can be set to the number of times the target object needs medication in a single day. The collection count for straight walking information can be the number of straight steps the wearable device collects from the target object. The collection count can be the same as the target object's step count. For example, for each step the target object takes, the wearable device will collect walking information once. The predetermined collection count threshold can be set in advance by healthcare professionals based on the target object's condition to define the number of collections required for each walking detection.

Based on this, both the predetermined collection cycle threshold and the predetermined collection count threshold can be set by a doctor according to the target object's medication requirements.

According to an embodiment of the present invention, other types of walking for the target object can include turning, etc.

According to an embodiment of the present invention, if the target object turns and the number of straight steps collected before the turn is greater than or equal to the predetermined collection count threshold, it can be determined that the target object has completed a single detection for the day.

If the target object turns and the number of straight steps collected before the turn is less than the predetermined collection count threshold, it can be determined that the single detection for the day has not been completed. Based on this, the wearable device can reset the step count and clear the cached walking information. When straight walking is detected again, new walking information is collected until the number of straight steps before the target object turns reaches or exceeds the predetermined collection count threshold.

If the target object fails to perform continuous straight walking before the turn during the detection period, and the number of straight steps is less than the predetermined collection count threshold, it will be determined that this walking detection has failed.

According to an embodiment of the present invention, if the target object's daily collection cycle count is greater than or equal to the predetermined collection cycle threshold, it can be determined that the target object has completed all walking detections for the day.

If the target object's daily collection cycle count is less than the predetermined collection cycle threshold, it can be determined that the target object has not completed all walking detections for the day.

Based on this, if the collection count for the walking information is greater than or equal to the predetermined collection count threshold, and the collected walking information is of sufficient quality to support subsequent data calculation and analysis, the information collection for that gait detection period can be stopped to reduce the power consumption of the wearable device. Turning and later information can be deleted, retaining only the walking information before the turn to improve the effective utilization of storage space.

According to an embodiment of the present invention, when the target object completes the last walking detection for the day, it can be detected that the target object transitions from straight walking to another type of walking, and the number of straight walking collections is greater than or equal to the predetermined collection count threshold, with the daily collection cycle count for straight walking also being greater than or equal to the predetermined collection cycle threshold.

According to an embodiment of the present invention, if the target object completes the last detection task for the day, and the daily collection cycle count for straight walking is less than the predetermined collection cycle threshold, it can be determined that the walking detection for that day has failed. All the walking information collected that day can be deleted to avoid wasting the wearable device's resources on invalid walking information.

According to an embodiment of the present invention, by using confirmation information, the target object's motion state, walking condition, the number of times walking information is collected, and the number of daily collection cycles, the next step can be adaptively determined based on the actual situation of the target object. This helps avoid wasting the wearable device's battery resources, extending its standby time. Additionally, by avoiding unnecessary operations on the wearable device, its computational power can be fully utilized to process only the necessary walking information, thus avoiding the processing of invalid walking information and reducing the wearable device's resource consumption.

Moreover, by sending only the gait parameters to the mobile communication terminal, the amount of information sent to the terminal is reduced, which improves the information processing efficiency of the mobile communication terminal and conserves its resources.

According to an embodiment of the present invention, the walking information includes the first segment, the second segment, and the third segment of walking information, which are sequentially collected in order. The gait information processing method further includes: determining the target object's first straight walking condition information based on the first and third segments of walking information. In response to the first straight walking condition information indicating a transition from straight walking to turning, the second straight walking condition information is determined based on the second segment of walking information. Based on the second straight walking condition information, it is determined that the target object has transitioned from straight walking to another type of walking.

According to an embodiment of the present invention, the first straight walking condition information can include the first straight walking condition information indicating a turn and the first straight walking condition information indicating straight walking.

According to an embodiment of the present invention, the second straight walking condition information can include the second straight walking condition information indicating a turn and the second straight walking condition information indicating straight walking.

According to an embodiment of the present invention, the first segment, the second segment, and the third segment of walking can be obtained by dividing the total number of steps the target object took during a single detection into three equal parts. For example, if the target object walks 30 steps during a single detection, the first segment could consist of the first 10 steps, the second segment could consist of the middle 10 steps, and the third segment could consist of the last 10 steps. The first segment walking information includes the walking data for each step in the first segment, the second segment walking information includes the walking data for each step in the second segment, and the third segment walking information includes the walking data for each step in the third segment.

According to an embodiment of the present invention, by using the first straight walking condition information obtained from the first and second segments of walking information, the overall walking trend of the target object can be determined. Based on this, if the first straight walking condition information indicates that the target object is walking straight, the operation to determine the second straight walking condition information can be skipped, saving power consumption on the wearable device.

According to an embodiment of the present invention, if the second straight walking condition information indicates that the target object is walking straight, it can be determined that the target object has not transitioned from straight walking to another type of walking. If the second straight walking condition information indicates that the target object is turning, it can be determined that the target object has transitioned from straight walking to another type of walking.

When the first straight walking condition information indicates that the target object has transitioned from straight walking to turning, the second straight walking condition information can be determined based on the second segment of walking, which lies between the first and third segments. Since the second segment of walking is positioned between the first and third segments, it can better reflect the target object's turning trend in greater detail. Based on this, the second segment walking information can more accurately determine the target object's straight walking condition, improving the accuracy of identifying the target object's straight walking condition.

According to an embodiment of the present invention, the first segment of walking information includes the first step position coordinates, and the third segment of walking information includes the second step position coordinates. Based on the first and third segments of walking information, the target object's first straight walking condition information is determined, including: inputting the first step position coordinates into a line fitting function, and fitting to obtain the first fitted line. The first fitted line includes the first predicted position coordinates corresponding to the first step position coordinates, and the sum of the squared errors between the first predicted position coordinates and the first step position coordinates is minimized. The second step position coordinates are input into the same line fitting function, and fitting is performed to obtain the second fitted line. The second fitted line includes the second predicted position coordinates corresponding to the second step position coordinates, and the sum of the squared errors between the second predicted position coordinates and the second step position coordinates is minimized. When the sum of the first and second squared errors meets a predetermined error condition, the first straight walking condition information for the target object is determined based on the first slope of the first fitted line and the second slope of the second fitted line.

According to an embodiment of the present invention, the first step position coordinates and the second step position coordinates can both be coordinates in the Earth coordinate system. The first step position coordinates and the second step position coordinates can both be coordinates in the form of (x, y). The line fitting function can be y=kx+c, where kk is the slope of the line, and cc is the intercept of the line.

According to an embodiment of the present invention, the first predicted position coordinates can be the coordinates obtained from fitting the first fitted line. The second predicted position coordinates can be the coordinates obtained from fitting the second fitted line.

The first step position coordinates corresponding to the first predicted position coordinates can be the first step position coordinates that are closest to the first predicted position coordinates.

The second step position coordinates corresponding to the second predicted position coordinates can be the second step position coordinates that are closest to the second predicted position coordinates.

According to an embodiment of the present invention, the first squared error can be determined based on the squared distance between the first predicted position coordinates and the first step position coordinates. The second squared error can be determined based on the squared distance between the second predicted position coordinates and the second step position coordinates.

According to an embodiment of the present invention, if the first squared error and the second squared error are both smaller than a predetermined error threshold, it can be determined that the target object's first and third segments of walking are straight walking, and the first and second squared errors meet the predetermined error condition.

According to an embodiment of the present invention, if at least one of the first squared error and the second squared error is greater than or equal to the predetermined error threshold, it can be determined that the target object has turned during at least one of the first and third segments of walking, and the first and second squared errors do not meet the predetermined error condition.

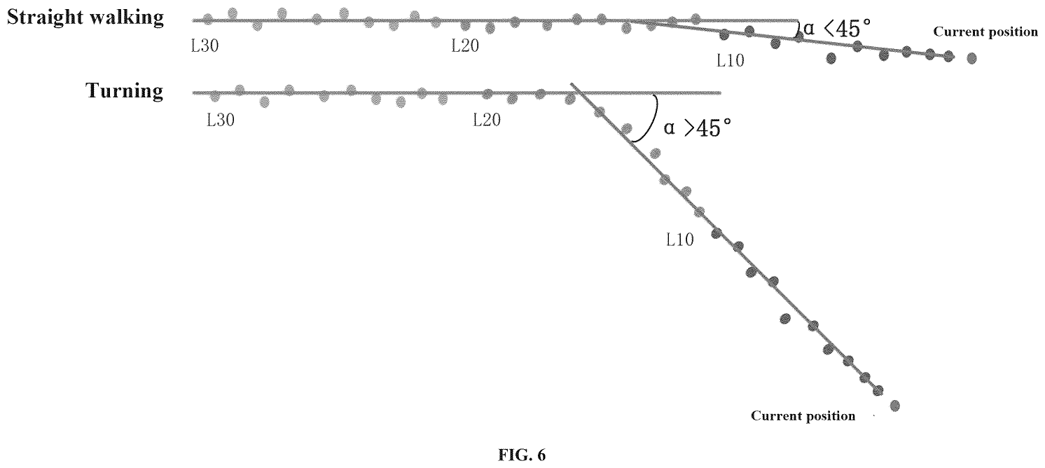

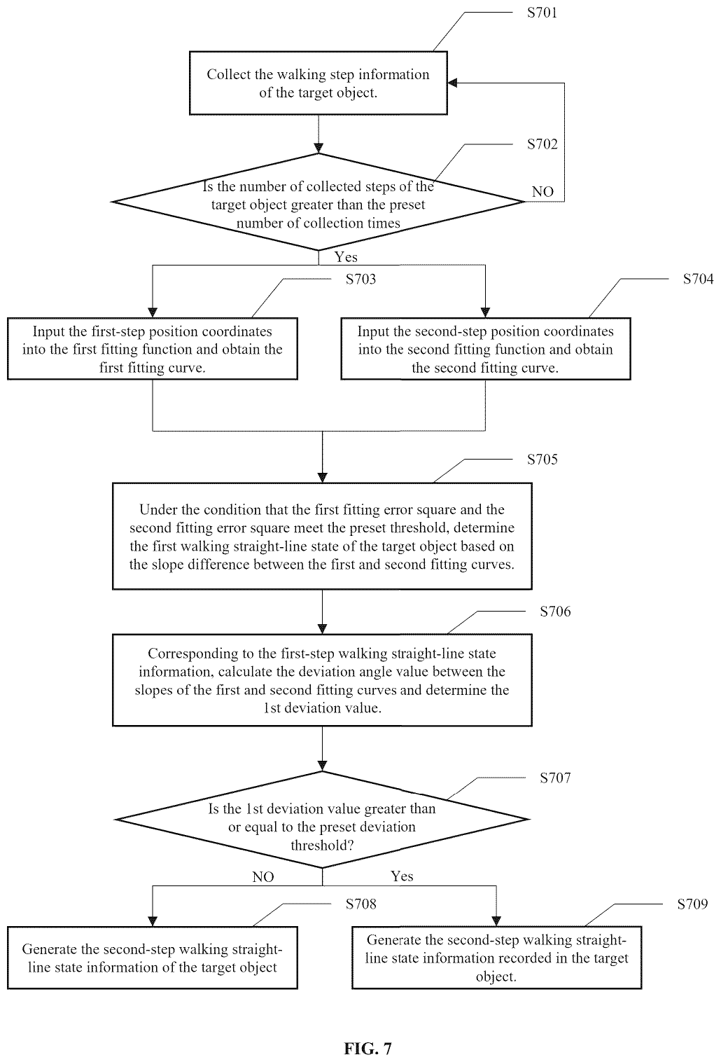

FIG. 6 shows a schematic diagram of the turn detection according to an embodiment of the present invention.

According to an embodiment of the present invention, the predetermined collection count threshold can be 30, but is not limited to this. As shown in FIG. 6, after the target object has taken 30 steps, the overall walking condition of the target object can be determined to be either straight walking or turning based on the first 30 steps of walking information.

Data from the foot contact points can be taken from two sets of data: one from the 30ₜₕ step to the 21ₛₜ step before the current position, and the other from the 10ₜₕ step to the 1ₛₜ step before the current position. The data from the 30ₜₕ step to the 21ₛₜ step can correspond to the first segment of walking, and the data from the 10ₜₕ step to the 1ₛₜ step can correspond to the third segment of walking.

Based on this, the first and third segments of walking can each have ten-step position coordinates. The first step position coordinates of the first walking segment can be represented as L₃₀˜L₂₁. The second step position coordinates of the third segment of walking can be represented as L₁₀˜L₁. Here, the i^{th} step position coordinates Lᵢ is represented by the two-dimensional coordinates (xᵢ, yᵢ).

According to an embodiment of the present invention, the least squares method can be used to perform linear fitting on the first-step position coordinates and the second-step position coordinates. Taking the position data from L₃₀˜L₂₁ as an example, the line fitting function uses the slope-intercept form equation: y=kx+c where is k the slope of the line and c is the intercept of the line.

Thus, the fitted line can be expressed as:

$${\hat{y}}_{i} = {\hat{k}}_{1} {x}_{i} + {\hat{c}}_{1} \qquad ( 21 )$$

\hat{k}₁ is the slope of the fitted line. ĉ₁ is the intercept. ŷᵢ is the corresponding value of xᵢ on the fitted line. In matrix form, this is represented as:

$$\hat{y} = X θ \qquad ( 22 )$$

where the original data matrix

$$X = [ \begin{matrix}{x}_{1} & 1 \\ {x}_{2} & 1 \\ ⋮ & ⋮ \\ {x}_{10} & 1\end{matrix} ] ,$$

the parameter matrix to be determined

$$θ = [ \begin{matrix}{\hat{k}}_{1} \\ {\hat{c}}_{1}\end{matrix} ] ,$$

and the fitted data matrix

$$\hat{y} = [ \begin{matrix}{\hat{y}}_{1} \\ {\hat{y}}_{2} \\ ⋮ \\ {\hat{y}}_{10}\end{matrix} ] .$$

The objective function is defined as the sum of the squared errors between the fitted cur and the actual positions, given by:

$${J}_{1} = \frac{1}{2} {( \hat{y} - y )}^{T} ( \hat{y} - y ) \qquad ( 23 )$$

Where ŷ represents the fitted values and y represents the actual values.

The matrix form of the first fitted line is substituted into the calculation formula for the first sum of squared errors to obtain:

$${J}_{1} = \frac{1}{2} {( X θ - y )}^{T} ( X θ - y ) \qquad ( 24 )$$

where J₁ represents the first sum of squared errors. y represents the first step position coordinates. The matrix

$$X = [ \begin{matrix}{x}_{1} & 1 \\ {x}_{2} & 1 \\ ⋮ & ⋮ \\ {x}_{10} & 1\end{matrix} ]$$

can represent the actual data, which includes the first step position coordinates. The parameter matrix

$$θ = [ \begin{matrix}{\hat{k}}_{1} \\ {\hat{c}}_{1}\end{matrix} ]$$

is to be solved.

To obtain the value of θ that minimizes the first sum of squared errors J₁, the partial derivative of J₁ with respect to θ can be taken and set to zero, that is:

$$\frac{\partial }{\partial θ} {J}_{1} = {X}^{T} ( X θ - y ) = 0 \qquad ( 25 )$$

where J₁ represents the first sum of squared errors. y represents the first step position coordinates. The matrix

$$X = [ \begin{matrix}{x}_{1} & 1 \\ {x}_{2} & 1 \\ ⋮ & ⋮ \\ {x}_{10} & 1\end{matrix} ]$$

can represent the actual data, which includes the first step position coordinates. The parameter matrix

$$θ = [ \begin{matrix}{\hat{k}}_{1} \\ {\hat{c}}_{1}\end{matrix} ]$$

is to be solved.

The solution is obtained as follows:

$$θ = [ \begin{matrix}{\hat{k}}_{1} \\ {\hat{c}}_{1}\end{matrix} ] = {( {X}^{T} Y )}^{- 1} {X}^{T} y \qquad ( 26 )$$

y represents the first step position coordinates. The matrix

$$X = [ \begin{matrix}{x}_{1} & 1 \\ {x}_{2} & 1 \\ ⋮ & ⋮ \\ {x}_{10} & 1\end{matrix} ]$$

can represent the actual data, which includes the first step position coordinates. The parameter matrix

$$θ = [ \begin{matrix}{\hat{k}}_{1} \\ {\hat{c}}_{1}\end{matrix} ]$$

is to be solved.
from which the fitted line for the L₁₀˜L₁ data can be obtained as ŷᵢ=\hat{k}₁xᵢ+ĉ₁. At this point, the minimum sum of squared errors [symbol] can be calculated and compared with an experimentally determined threshold J_{threshold}. If [symbol] <J_{threshold}, the movement is determined to be straight. Similarly, the fitted line for the L₃₀˜L₂₁ data can be expressed as ŷᵢ=\hat{k}₂xᵢ+ĉ₂ with the corresponding minimum sum of squared errors [symbol] , which can also be used to judge whether the movement is straight. If both sets of data indicate straight-line movement, the angle α between the two fitted lines is calculated. The direction vectors of the two lines are \vec{s}₁=(\hat{k}₁, 1) and \vec{s}₂=(\hat{k}₂, 1). The angle α between them is given by

$$α = arccos \frac{{\vec{s}}_{1} {\vec{s}}_{2}}{‖ {\vec{s}}_{1} ‖ ‖ {\vec{s}}_{2 ‖}} \qquad ( 27 )$$

When the angle α is greater than 45°, the overall walking trend of the target object can be determined to be a turning trend, and the first straight walking condition information representing the target object's turn is generated.

When the angle α is less than or equal to 45°, the overall walking trend of the target object can be determined to be a straight walking trend, and the first straight walking condition information representing the target object's straight walking is generated.

According to an embodiment of the present invention, by minimizing the first sum of squared errors between the first predicted position coordinates and the first step position coordinates, an accurate first fitted line can be obtained. Similarly, by minimizing the second sum of squared errors between the second predicted position coordinates and the second step position coordinates, an accurate second fitted line can be obtained. Based on this, the accurate first straight walking condition information can be determined using the first and second fitted lines.

Furthermore, the first straight walking condition information is determined only when the first and second sums of squared errors meet the predetermined error condition, which avoids unnecessary power consumption of the wearable device and improves its battery life.

According to an embodiment of the present invention, the second segment of walking information includes Q yaw angle values, where Q is a positive integer greater than 1. In response to the first straight walking condition information indicating a transition from straight walking to turning, the second straight walking condition information is determined based on the second segment of walking information, including: determining the difference between the qₜₕ yaw angle value and the q+1ₜₕ yaw angle value, obtaining Q−1 differences, where qqq is a positive integer less than Q In response to the sum of the Q−1 differences being greater than or equal to a predetermined difference threshold, the second straight walking condition information representing the target object's turn is generated. In response to the sum of the Q−1 differences being less than the predetermined difference threshold, the second straight walking condition information representing the target object's straight walking is generated.

According to an embodiment of the present invention, the second segment of walking for the target object may include Q steps, where Q can be less than III. The second segment of walking information for the target object may include Q pieces of walking information corresponding to the Q steps. Each piece of walking information may include a yaw angle value. Thus, the Q pieces of walking information can include Q yaw angle values.

According to an embodiment of the present invention, the yaw angle difference between two adjacent steps can be calculated as Δβ_{q}=β_{q+1}−β_{q}, thereby obtaining Q−1 differences. Here, Δβ_{q} represents the qₜₕ yaw angle value, and β_{q+1} represents the q+1ₜₕ yaw angle value.

The Q−1 differences can be summed, as shown in the following formula:

$${Δβ}_{sum} = \vert {\sum }_{q = 1}^{Q - 1} Δ {β}_{q} \vert \qquad ( 28 )$$

where Δβₛᵤₘ represents the sum of the Q−1 differences, and Δβ_{q} represents the difference between the qₜₕ yaw angle value and the q+1ₜₕ yaw angle value.

When Δβₛᵤₘ≥45°, it can be determined that the target object has turned during the second segment of walking, and the second straight walking condition information representing the target object's turn is generated.

When Δβₛᵤₘ<45°, it can be determined that the target object is walking straight during the second segment of walking, and the second straight walking condition information representing the target object's straight walking is generated. Here, 45° can be the predetermined difference threshold. It should be noted that this predetermined difference threshold can be set based on specific requirements and is not limited to 45°.

According to an embodiment of the present invention, by calculating the difference between yaw angle values one by one, the straight walking condition of the target object during the second segment of walking can be determined, thereby improving the accuracy of determining the target object's straight walking condition from a detailed perspective.

According to an embodiment of the present invention, if the second straight walking condition information indicates a turn for the target object, the third straight walking condition information for the target object can be determined based on the qₜₕ step in the Q steps, the first fitted line, and the second fitted line.

The third straight walking condition information can include information indicating a transition from straight walking to turning, as well as information indicating that the target object has not transitioned from straight walking to turning.

If the third straight walking condition information indicates that the target object has turned, it is determined that the target object has transitioned from straight walking to another type of walking. If the third straight walking condition information indicates straight walking, it is determined that the target object has not transitioned from straight walking to another type of walking.

According to an embodiment of the present invention, the qₜₕ step in the Q steps can include multiple position coordinates for the q-th step. These q position coordinates can be input into the line fitting function to obtain the fitted line for the qₜₕ step.

The first angle between the first fitted line and the fitted line for the qₜₕ step, as well as the second angle between the second fitted line and the fitted line for the qₜₕ step, can be determined. The opening of the first angle may be towards the direction of the third segment of walking of the target object, while the opening of the second angle may be towards the direction of the first segment of walking of the target object. The first angle can be calculated based on the slope of the first fitted line and the slope of the qₜₕ step's fitted line, and the second angle can be calculated similarly.

If the sum of the values of the first and second angles is greater than or equal to the predetermined angle threshold, the qₜₕ step can be determined to be a target step.

If the sum of the values of the first and second angles is less than the predetermined angle threshold, the qₜₕ step can be determined to be a non-target step.

If the proportion of target steps among the Q steps is greater than or equal to a predetermined threshold, the third straight walking condition information representing the target object's turn is generated.

If the proportion of target steps among the Q steps is less than the predetermined threshold, the third straight walking condition information representing the target object's straight walking is generated.

The predetermined angle threshold and the predetermined ratio threshold can be set according to specific requirements, and are not limited by the present invention.

Based on this, by using the first and second angles, the straight walking condition of the target object can be determined for each step in the second segment of walking, from a more detailed perspective compared to the second straight walking condition information, thereby improving the accuracy of determining the target object's walking condition.

FIG. 7 shows a flowchart of a method for generating second straight walking condition information according to an embodiment of the present invention.

As shown in FIG. 7, the method for generating second straight walking condition information in this embodiment includes operations S701 to S709.

In operation S701, the walking information of the target object's straight walking is collected.

In operation S702, is the collection count for the target object's straight walking information greater than the predetermined collection count threshold? If yes, then perform operations S703 and S704; if no, return to perform operation S701.

In operation S703, input the first step position coordinates into the line fitting function to obtain the first fitted line.

In operation S704, input the second step position coordinates into the line fitting function to obtain the second fitted line.

In operation S705, if the first and second sums of squared errors meet the predetermined error condition, determine the first straight walking condition information of the target object based on the first slope of the first fitted line and the second slope of the second fitted line.

In operation S706, in response to the first straight walking condition information indicating that the target object has transitioned from straight walking to turning, determine the difference between the iₜₕ yaw angle value and the i+1ₜₕ yaw angle value among the I yaw angle values, obtaining I−1 differences.

In operation S707, is I−1 differences greater than or equal to the predetermined difference threshold? If no, perform operation S708; if yes, perform operation S709.

In operation S708, generate the second straight walking condition information representing the target object's straight walking.

In operation S709, generate the second straight walking condition information representing the target object's turn.

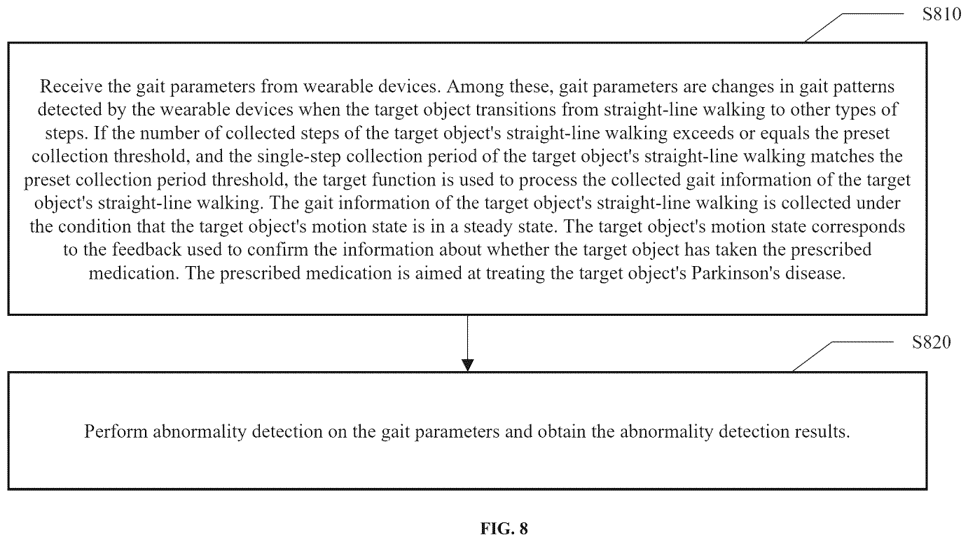

FIG. 8 shows a flowchart of a gait information processing method according to another embodiment of the present invention.

As shown in FIG. 8, the gait information processing method of this embodiment can be applied to a mobile communication terminal. The method includes operations S810 to S820.

In operation S810, receive gait parameters from a wearable device, where the gait parameters are obtained by the wearable device in response to detecting that the target object has transitioned from straight walking to another type of walking, with the collection count of the target object's straight walking information being greater than or equal to a predetermined collection count threshold, and the daily collection cycle count of the straight walking information being greater than or equal to a predetermined collection cycle threshold. The gait information of the target object's straight walking is collected when the target object is in a walking state, and the motion state of the target object is detected in response to receiving confirmation information indicating that the target object has used the specified medication.

In operation S820, perform anomaly detection on the gait parameters to obtain the anomaly detection result.

According to an embodiment of the present invention, a fixed detection period for walking detection can be preset by a doctor. The interval between these periods can be 0 hours, 1 hour, 2 hours, 3 hours, 4 hours, etc.

For example, at a target time before the detection period, the mobile communication terminal can provide a medication reminder to the target object. For example, 5 minutes before the detection period, the mobile communication terminal can provide a reminder to the target object by ringing.

For example, when the detection period arrives and no instruction indicating the effectiveness of the medication reminder has been received, the mobile communication terminal can prompt the target object at intervals of a first predetermined duration. For instance, if the detection period has arrived but no confirmation information indicating the effectiveness of the medication reminder has been received, the mobile communication terminal can ring once every 5 minutes. If the detection period arrives and no instruction from the target object to cancel the ringing has been received, it can be determined that the instruction indicating the effectiveness of the medication reminder has not been received. For example, the instruction indicating the effectiveness of the medication reminder can be an instruction to cancel the ringing. If the instruction indicating the target object cancels the ringing is received, it can be determined that the reminder is effective.

For example, if the instruction indicating the effectiveness of the medication reminder is received, and no confirmation information indicating that the target object has used the target medication is received within a second predetermined duration, the target object can be prompted to confirm the medication. The second predetermined duration can be 2 minutes, but it is not limited to this. For example, the target object can be reminded to confirm the medication by ringing.

According to an embodiment of the present invention, by using the confirmation information, the target object's motion state, walking condition, the number of times walking information is collected, and the number of daily collection cycles, the next step can be adaptively determined based on the actual situation of the target object. This helps avoid wasting the wearable device's battery resources, extending its standby time. Additionally, by avoiding unnecessary operations on the wearable device, its computational power can be fully utilized to process the necessary walking information.

Furthermore, by receiving gait parameters from the wearable device, the amount of information received by the mobile communication terminal is reduced, which improves the information processing efficiency of the mobile communication terminal and conserves its resources.

According to an embodiment of the present invention, performing anomaly detection on the gait parameters to obtain the anomaly detection result includes: responding to the number of days of collected walking information being equal to a predetermined number of days threshold, and using the first gait parameter anomaly detection algorithm to process the gait parameters, thereby obtaining the first anomaly detection result corresponding to the trend of the gait parameters. Using the second gait parameter anomaly detection algorithm to process the gait parameters results in the second anomaly detection result corresponding to the deviation of the gait parameters.

According to an embodiment of the present invention, the trend of gait parameters can refer to the trend in gait parameters across MMM medication periods within a single day. The deviation of gait parameters can refer to the deviation among gait parameters during KKK detection cycles within a single medication period.

According to an embodiment of the present invention, the mobile communication terminal can determine the number of days corresponding to the collected walking information based on the number of received gait parameters. If the number of days is less than the predetermined number of days threshold, it can be determined that the target object has not collected sufficient gait parameters, and the target object can continue to be reminded daily to take the medication and undergo detection.

According to an embodiment of the present invention, if the number of days is equal to the predetermined number of days threshold, it can be determined that the target object has collected sufficient gait parameters. Accordingly, the first gait parameter anomaly detection algorithm can be used to process the gait parameters to obtain the first anomaly detection result corresponding to the trend of the gait parameters. Additionally, the second gait parameter anomaly detection algorithm can be used to process the gait parameters to obtain the second anomaly detection result corresponding to the deviation of the gait parameters.

According to an embodiment of the present invention, by using the first gait parameter anomaly detection algorithm and the second gait parameter anomaly detection algorithm, the first anomaly detection result corresponding to the trend of the gait parameters and the second anomaly detection result corresponding to the deviation of the gait parameters can be obtained. This allows the determination of abnormal conditions in gait parameters from multiple aspects, thereby improving the accuracy of identifying abnormal gait parameters.

According to an embodiment of the present invention, there are M sets of gait parameters, corresponding to the M medication periods of the target object in a single day. The M sets of gait parameters are arranged sequentially in chronological order, where M is a positive integer greater than 1. Using the first gait parameter anomaly detection algorithm to process the gait parameters results in the first anomaly detection result corresponding to the trend of the gait parameters, which includes: calculating the (m−1)ₜₕ first-order forward difference value based on the (m−1)ₜₕ and mₜₕ sets of gait parameters among the M sets of gait parameters, where mmm is a positive integer greater than 1 and less than or equal to M. Based on the (m−2)ₜₕ and (m−1)ₜₕ first-order forward difference values among the M−1 first-order forward difference values, the (m−2)ₜₕ second-order forward difference value is calculated. If the M−2 second-order forward difference values are greater than the predetermined difference threshold, the first anomaly detection result indicating that no abnormal condition exists in the gait parameters is generated. If the M−2 second-order forward difference values are less than or equal to the predetermined difference threshold, the first anomaly detection result indicating an abnormal condition in the gait parameters is generated.

According to an embodiment of the present invention, based on clinical observations of Parkinson's patients after taking medication, the effect of the medication on gait regulation gradually increases within 2 hours after taking the medication, reaches a peak around 2 hours, and then gradually decreases. Therefore, there are two types of trends in the corresponding gait parameters. The trends can be classified into Type I and Type II s to the efficacy pattern of the target medication.

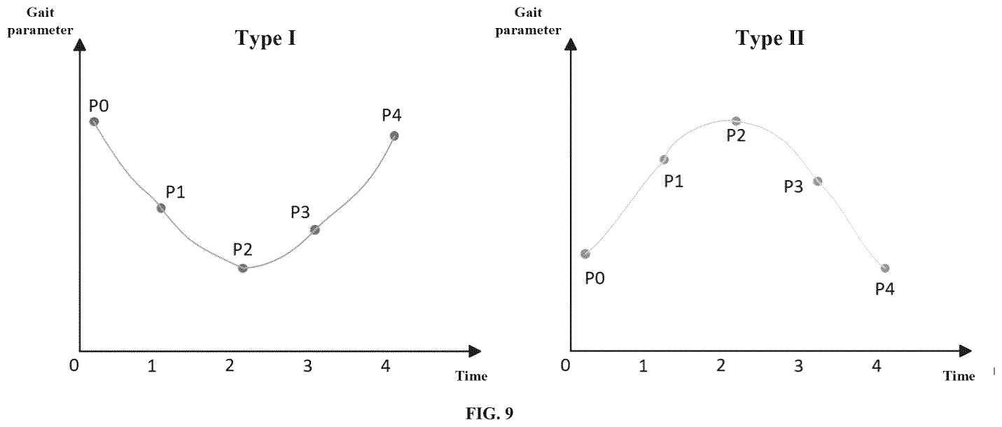

FIG. 9 shows a schematic diagram of the normal variation curve of gait parameters after medication according to an embodiment of the present invention.

As shown in FIG. 9, the horizontal axis represents the time after taking medication, and the vertical axis represents the value of gait parameters. P₀˜P₄ respectively represent the gait parameters at different time points tᵢ.

For Type I, defined as a concave function ƒ₁(t), the first forward difference is Δƒ₁(tᵢ)=ƒ₁(tᵢ₊₁)−ƒ₁(tᵢ), where i=0, 1, 2, 3. The second forward difference is Δ²ƒ₁(tᵢ)=Δƒ₁(tᵢ₊₁)−Δƒ₁(tᵢ), where i=0, 1, 2. If Δ²ƒ₁(tᵢ)>0 for all gait data, it indicates M−2 second-order forward difference values are greater than the predetermined difference threshold, it indicates compliance with the concave function definition, and thus it can be determined that the drug's effect on the target object's gait follows the normal pattern. Otherwise, it can be determined that there is an anomaly in the target object's gait parameters.

For Type II, defined as a concave function ƒ₂(t), the first forward difference is Δƒ₂(tᵢ)=ƒ₂(tᵢ₊₁)−ƒ₂(tᵢ), where i=0, 1, 2, 3. The second forward difference is Δ²ƒ₂(tᵢ)=Δƒ₂(tᵢ₊₁)−Δƒ₂(t), where i=0, 1, 2. If Δ²ƒ₂(tᵢ)>0 for all gait data, it indicates M−2 second-order forward difference values are lower than the predetermined difference threshold, it indicates compliance with the concave function definition, and thus it can be determined that the drug's effect on the target object's gait follows the normal pattern. Otherwise, it can be determined that there is an anomaly in the target object's gait parameters. 0 can represent the aforementioned predetermined difference threshold, which can be set in advance by a doctor.

Based on this, the first anomaly detection result can be generated from the 20 gait parameters of the target object's two feet, using the aforementioned first gait parameter anomaly detection algorithm.

According to an embodiment of the present invention, by sequentially calculating the first-order forward difference values between gait parameters, and then calculating the second-order forward difference values based on the first-order forward difference values, the variation pattern of the gait parameters over the target object's M medication periods can be accurately determined, leading to an accurate first anomaly detection result.

According to an embodiment of the present invention, the mₜₕ set of gait parameters includes K gait parameters corresponding to K detection cycles. Using the second gait parameter anomaly detection algorithm to process the gait parameters, the first anomaly detection result corresponding to the deviation of the gait parameters is obtained, which includes: determining the statistical values of the K gait parameters, and determining the K differences between the statistical values and the K gait parameters. If the K differences are less than or equal to the predetermined difference threshold, the second anomaly detection result indicating no abnormal condition in the gait parameters is generated. If the K differences are greater than the predetermined difference threshold, the second anomaly detection result indicating an abnormal condition in the gait parameters is generated.

According to an embodiment of the present invention, the statistical values of the K gait parameters can include the mean value of the K gait parameters, among other statistics.

According to an embodiment of the present invention, the gait parameters calculated from the gait information of the jₜₕ walking detection on the iₜₕ day can be represented as P_{i,j}. The average value of the gait parameters P̅ⱼ from the jₜₕ walking detection over one detection cycle can be calculated as follows:

$${\overline{P}}_{j} = \frac{1}{N} {\sum }_{i = 1}^{K} {P}_{i , j} \qquad ( 29 )$$

Where K can represent the number of detection cycles within a single medication period. K can be 7.

Based on this, the deviation of the gait parameters from the mean for the jₜₕ walking detection on each day can be calculated as P̅ⱼ, that is, the aforementioned difference, as shown below:

$${Δ}_{i , j} = \frac{\vert {P}_{i , j} - {\overline{P}}_{j} \vert}{{\sum }_{i = 1}^{K} \vert {P}_{i , j} - {\overline{P}}_{j} \vert} \qquad ( 30 )$$

Where P_{i,j} can represent the gait parameters calculated from the gait information of the jₜₕ walking detection on the iₜₕ day. P̅ⱼ can also represent the average value of the gait parameters from the jₜₕ walking detection over one detection cycle. K can be the number of detection cycles within a single medication period.

If the deviation is greater than the predetermined difference threshold, i.e., Δ_{i,j}>γ, the second anomaly detection result indicating an abnormal condition in the gait parameters is generated.

If the deviation is less than or equal to the predetermined difference threshold, i.e., Δ_{i,j}<γ, the second anomaly detection result indicating that the gait parameters are within normal condition is generated.

According to an embodiment of the present invention, by calculating the statistical values of K gait parameters and the differences between the K gait parameters, the fluctuation status of the gait parameters within a single detection cycle can be accurately determined.

According to an embodiment of the present invention, the gait information processing method further includes: in response to both the first and second anomaly detection results indicating that there is no abnormal condition in the gait parameters, sending the gait parameters to the server for storage. In response to at least one of the first and second anomaly detection results indicating that there is an abnormal condition in the gait parameters, sending an information request to the wearable device to obtain the walking information corresponding to the gait parameters with abnormal conditions stored in the wearable device, and then sending the walking information corresponding to the abnormal gait parameters to the server for storage.

According to an embodiment of the present invention, the wearable device can transmit the cached walking information with abnormal conditions to the body area network gateway, which is the mobile communication terminal.

After the mobile communication terminal receives the walking information with abnormal conditions, it can send the walking information with abnormal conditions to the server.

The server can receive and store the walking information with abnormal conditions uploaded by the mobile communication terminal. The server can send this information to the doctor's terminal so that the doctor can calculate and analyze the walking information with abnormal conditions. This allows the doctor to make adjustments to the target object, such as adjusting the medication dose and timing. As a result, non-abnormal walking information is not processed, thus conserving resources and improving information processing efficiency.

According to an embodiment of the present invention, the wearable device, mobile communication terminal, and server adaptively identify and manage data service demands for Parkinson's disease home medication management systems, improving the target object's user experience, doctor's diagnostic accuracy, and the system's data service performance.

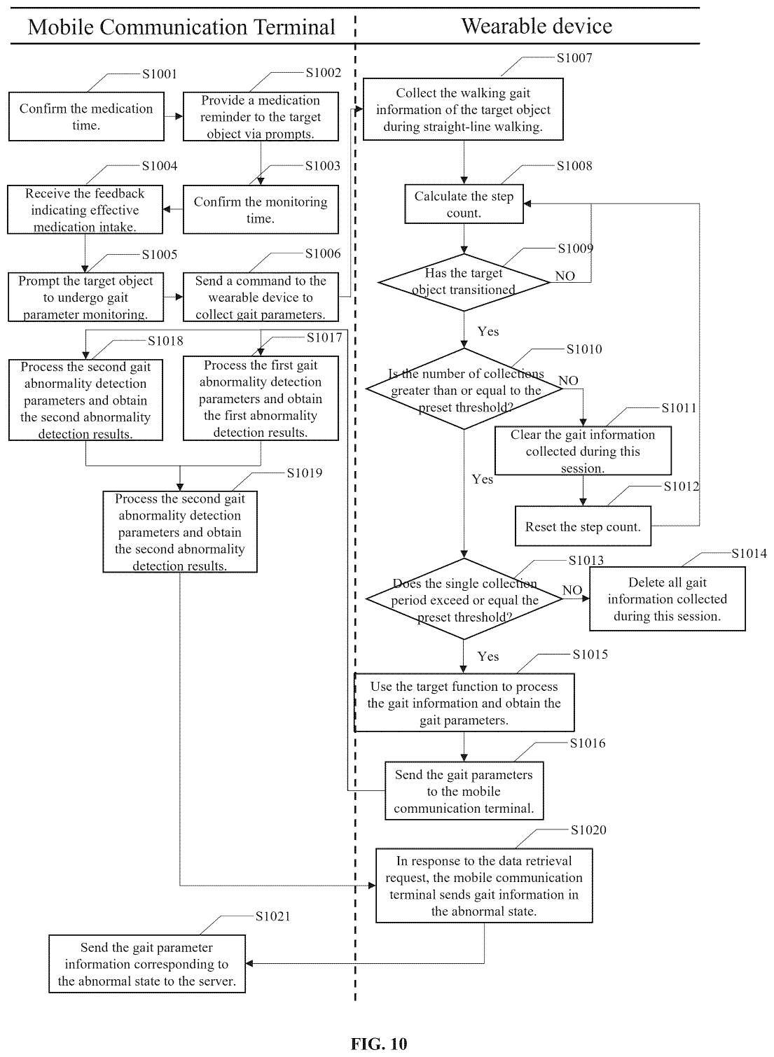

FIG. 10 shows a schematic diagram of the gait information processing method according to another embodiment of the present invention.

As shown in FIG. 10, the gait information processing method of this embodiment includes operations S1001 to S1021. Among them, operations S1001 to S1006, S1017 to S1019, and S1021 are executed by the mobile communication terminal, while operations S1007 to S1016 and S1020 are executed by the wearable device.

In operation S1001, it is determined that the medication time has arrived.

In operation S1002, a medication reminder is sent to the target object via ringing.

In operation S1003, an instruction indicating the effectiveness of the medication reminder is received.

In operation S1004, it is determined that the detection time has arrived.

In operation S1005, the target object is prompted to perform gait detection via ringing.

In operation S1006, a walking information collection command is sent to the wearable device.

In operation S1007, the straight walking information of the target object is collected.

In operation S1008, the number of steps is calculated.

In operation S1009, is the target object turning? If yes, execute operation S1010; if no, return to execute operation S1008.

In operation S1010, is the collection count greater than or equal to the predetermined collection count threshold? If yes, execute operation S1013; if no, execute operation S1011.

In operation S1011, the collected walking information is cleared.

In operation S1012, the step count is reset.

In operation S1013, is the daily collection cycle count greater than or equal to the predetermined collection cycle threshold? If yes, execute operation S1015; if no, execute operation S1014.

In operation S1014, all walking information collected on that day is deleted.

In operation S1015, the walking information is processed using a target function to obtain gait parameters.

In operation S1016, gait parameters are sent to the mobile communication terminal.

In operation S1017, the first gait parameter anomaly detection algorithm is used to process the gait parameters and obtain the first anomaly detection result.

In operation S1018, the second gait parameter anomaly detection algorithm is used to process the gait parameters and obtain the second anomaly detection result.

In operation S1019, in response to at least one of the first or second anomaly detection results indicating an abnormal condition in the gait parameters, an information request is sent to the wearable device.

In operation S1020, in response to receiving the information request, walking information with abnormal conditions is sent to the mobile communication terminal.

In operation S1021, walking information corresponding to the abnormal gait parameters is sent to the server.

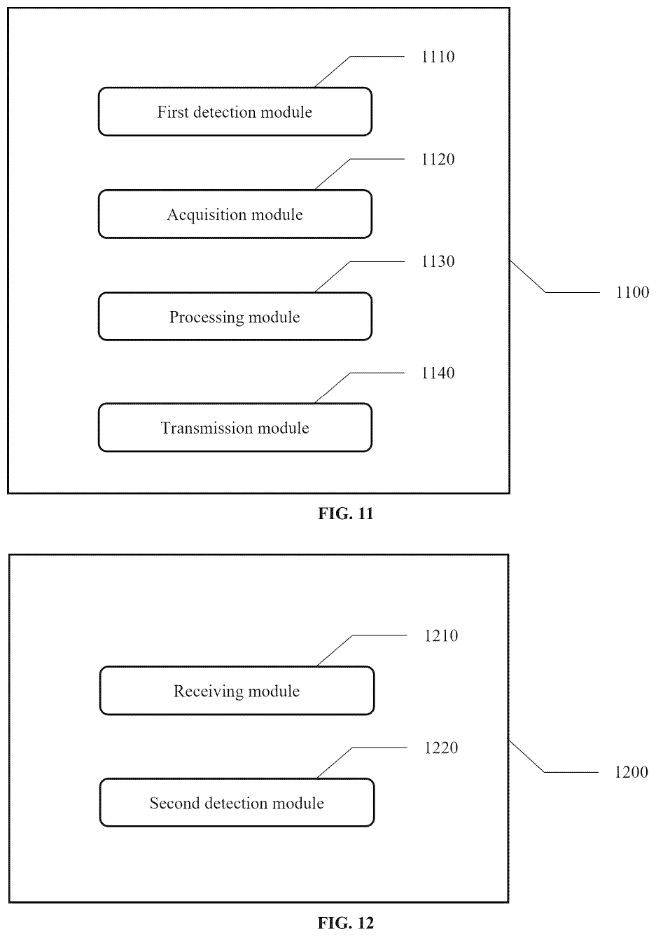

Based on the gait information processing method described above, the present invention also provides a wearable device. The structure of this wearable device will be described in detail in conjunction with FIG. 11.

FIG. 11 shows a block diagram of the wearable device according to an embodiment of the present invention.

As shown in FIG. 11, the wearable device 1100 in this embodiment includes a first detection module 1110, an acquisition module 1120, a processing module 1130, and a transmission module 1140.

The first detection module 1110 is used to detect the motion state of the target object in response to receiving confirmation information indicating that the target object has used the target medication. In one embodiment, the first detection module 1110 can perform the operation described earlier in Operation S210, which will not be repeated here.

The acquisition module 1120 is used to collect the target object's straight walking information when the motion state of the target object is walking. In one embodiment, the acquisition module 1120 can perform the operation described earlier in Operation S220, which will not be repeated here.

In operation S1006, the processing module 1130 is used to process the walking information of the target object's straight walking, using the target function, to obtain gait parameters. The processing module 1130 responds to detecting that the target object has transitioned from straight walking to another type of walking, with the collection count of the target object's straight walking greater than or equal to the predetermined collection count threshold, and the daily collection cycle count of the straight walking information greater than or equal to the predetermined collection cycle threshold. In one embodiment, the processing module 1130 can perform operation S230 described earlier.

The transmission module 1140 is used to send gait parameters to the mobile communication terminal, so that the mobile communication terminal can perform anomaly detection on the gait parameters. In one embodiment, the transmission module 1140 can perform operation S240 described earlier.

According to an embodiment of the present invention, the wearable device further includes a first determination module, a second determination module, a first generation module, and a second generation module. The first determination module is used to determine the first straight walking condition information of the target object based on the first and third segments of walking information. The second determination module, in response to the first straight walking condition information indicating that the target object has transitioned from straight walking to turning, determines the second straight walking condition information of the target object based on the second segment of walking information. The first generation module generates the second straight walking condition information indicating that the target object has transitioned from straight walking to another type of walking. The second generation module, in response to the sum of the I−1 differences being less than or equal to the predetermined difference threshold, generates the second straight walking condition information indicating that the target object is walking straight.

According to an embodiment of the present invention, the first determination module includes a first fitting submodule, a second fitting submodule, and a first determination submodule. The first fitting submodule inputs the first step position coordinates into the line fitting function to obtain the first fitted line. The first fitted line includes the first predicted position coordinates corresponding to the first step position coordinates, and the sum of the squared errors between the first predicted position coordinates and the first step position coordinates is minimized. The second fitting submodule inputs the second step position coordinates into the line fitting function to obtain the second fitted line. The second fitted line includes the second predicted position coordinates corresponding to the second step position coordinates, and the sum of the squared errors between the second predicted position coordinates and the second step position coordinates is minimized. The first determination submodule, when the first and second squared errors meet the predetermined error condition, determines the first straight walking condition information of the target object based on the first slope of the first fitted line and the second slope of the second fitted line.

According to an embodiment of the present invention, the second determination module includes a second determination submodule and a third determination submodule. The second determination submodule determines the difference between the qₜₕ and (q+1)ₜₕ yaw angle values among the Q yaw angle values, resulting in Q−1 differences, where q is a positive integer less than Q. The third determination submodule, in response to the sum of the Q−1 differences being greater than or equal to the predetermined difference threshold, determines the second straight walking condition information of the target object.

According to an embodiment of the present invention, any of the first detection module 1110, acquisition module 1120, processing module 1130, and transmission module 1140 can be combined into a single module, or any of the modules can be split into multiple modules. Alternatively, one or more of the functions of these modules can be combined with the functions of other modules and implemented in a single module. According to an embodiment of the present invention, at least one of the first detection module 1110, acquisition module 1120, processing module 1130, and transmission module 1140 can be partially implemented as hardware circuits, such as field-programmable gate arrays (FPGA), programmable logic arrays (PLA), system-on-chip (SoC), system-in-package (SiP), application-specific integrated circuits (ASIC), or implemented through other reasonable hardware or firmware methods such as integrated circuits or packaged circuits. Alternatively, at least one of the modules can be implemented as a computer program module, and when the computer program module is executed, it can perform the corresponding functionality.

FIG. 12 shows a block diagram of the mobile communication terminal according to an embodiment of the present invention.

As shown in FIG. 12, the mobile communication terminal 1200 in this embodiment includes a reception module 1210 and a second detection module 1220.

The reception module 1210 is used to receive gait parameters from the wearable device, where the gait parameters are obtained by the wearable device in response to detecting that the target object has transitioned from straight walking to another type of walking, with the collection count of the target object's straight walking being greater than or equal to the predetermined collection count threshold, and the daily collection cycle count of the straight walking information being greater than or equal to the predetermined collection cycle threshold. The gait information of the target object's straight walking is collected when the target object is in a walking state, and the target object's motion state is detected in response to receiving confirmation information indicating that the target object has used the target medication. In one embodiment, the reception module 1210 can perform operation S810 described earlier.

The second detection module 1220 is used to perform anomaly detection on the gait parameters and obtain the anomaly detection result. In one embodiment, the second detection module 1220 can perform operation S820 described earlier.

According to an embodiment of the present invention, the second detection module 1220 includes a first processing submodule and a second processing submodule. The first processing submodule is used, in response to the number of days corresponding to the collected walking information being equal to the predetermined number of days threshold, to process the gait parameters using the first gait parameter anomaly detection algorithm, obtaining the first anomaly detection result corresponding to the trend of the gait parameters. The second processing submodule is used to process the gait parameters using the second gait parameter anomaly detection algorithm, obtaining the second anomaly detection result corresponding to the deviation of the gait parameters.

According to an embodiment of the present invention, the first processing submodule includes a first calculation unit, a second calculation unit, a first generation unit, and a second generation unit. The first calculation unit is used to calculate the first-order forward difference value of the (m−1)ₜₕ and mₜₕ sets of gait parameters from the M sets of gait parameters, where mmm is a positive integer greater than 1 and less than or equal to M. The second calculation unit is used to calculate the second-order forward difference value of the (m−2)ₜₕ and (m−1)ₜₕ first-order forward difference values from the M−1 first-order forward difference values. The first generation unit is used, in response to the M−2 second-order forward difference values being greater than the predetermined difference threshold, to generate the first anomaly detection result indicating no abnormal condition in the gait parameters. The second generation unit is used, in response to the M−2 second-order forward difference values being less than or equal to the predetermined difference threshold, to generate the first anomaly detection result indicating an abnormal condition in the gait parameters.

According to an embodiment of the present invention, the second processing submodule includes a first determination unit, a second determination unit, a third generation unit, and a fourth generation unit. The first determination unit is used to determine the statistical values of the K gait parameters. The second determination unit is used to determine the K differences between the statistical values and the K gait parameters. The third generation unit is used, in response to the K differences being less than or equal to the predetermined difference threshold, to generate the second anomaly detection result indicating no abnormal condition in the gait parameters. The fourth generation unit is used, in response to the K differences being greater than the predetermined difference threshold, to generate the second anomaly detection result indicating an abnormal condition in the gait parameters.

According to an embodiment of the present invention, the second detection module 1220 further includes a first sending submodule and a second sending submodule. The first sending submodule is used, in response to both the first and second anomaly detection results indicating that there is no abnormal condition in the gait parameters, to send the gait parameters to the server for storage. The second sending submodule is used, in response to at least one of the first and second anomaly detection results indicating an abnormal condition in the gait parameters, to send an information request to the wearable device to obtain the walking information corresponding to the abnormal gait parameters cached in the wearable device, and to send the walking information corresponding to the abnormal gait parameters to the server for storage.

According to an embodiment of the present invention, any of the modules in the reception module 1210 and the second detection module 1220 can be merged into a single module, or any of the modules can be split into multiple modules. Alternatively, one or more of these modules can combine at least part of their functionality with at least part of the functionality of other modules and be implemented in a single module. According to an embodiment of the present invention, at least one of the reception module 1210 and the second detection module 1220 can be at least partially implemented as hardware circuits, such as field-programmable gate arrays (FPGA), programmable logic arrays (PLA), system-on-chip (SoC), system-in-package (SiP), application-specific integrated circuits (ASIC), or implemented by integrating or packaging circuits using any other reasonable hardware or firmware methods. Alternatively, one or more of these modules can be implemented as a computer program module, and when the computer program module is executed, it can perform the corresponding functionality.

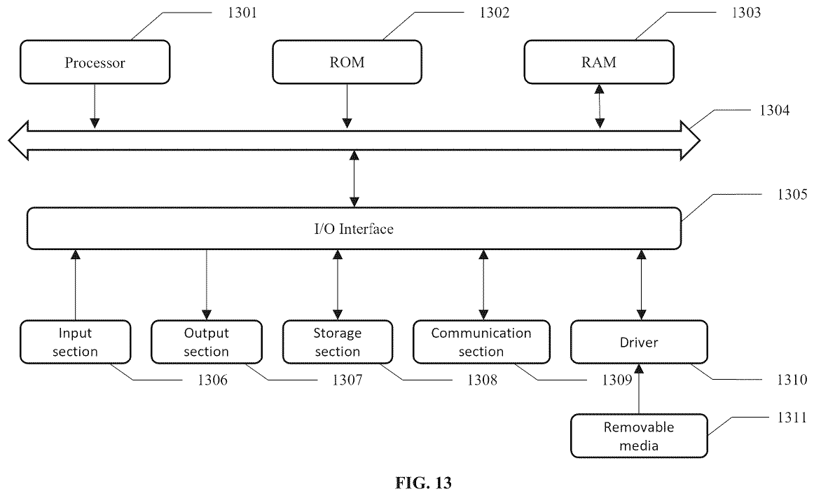

FIG. 13 shows a block diagram of an electronic device for implementing the gait information processing method according to an embodiment of the present invention.

As shown in FIG. 13, the electronic device 1300 according to an embodiment of the present invention includes a processor 1301, which can execute various appropriate actions and processes based on a program stored in the read-only memory (ROM) 1302 or a program loaded from the storage section 1308 into the random access memory (RAM) 1303. The processor 1301 can include, for example, a general-purpose microprocessor (such as a CPU), an instruction set processor, and/or associated chipsets, and/or a dedicated microprocessor (such as an application-specific integrated circuit (ASIC)), etc. The processor 1301 can also include onboard memory for caching purposes. The processor 1301 can include a single processing unit or multiple processing units to perform the various actions of the method flow according to the present invention.

The RAM 1303 stores various programs and data necessary for the operation of the electronic device 1300. The processor 1301, ROM 1302, and RAM 1303 are interconnected via bus 1304. The processor 1301 executes various operations of the method flow based on the program in ROM 1302 and/or RAM 1303. It should be noted that the program can also be stored in one or more storage devices other than ROM 1302 and RAM 1303. The processor 1301 can also execute the program stored in the one or more storage devices to perform the various operations of the method flow according to the embodiment of the present invention.

According to an embodiment of the present invention, the electronic device 1300 also includes an input/output (I/O) interface 1305, which is also connected to the bus 1304. The electronic device 1300 can further include one or more of the following components connected to the input/output (I/O) interface 1305: an input section 1306, such as a keyboard, mouse, etc.; an output section 1307, such as a cathode ray tube (CRT), liquid crystal display (LCD), speakers, etc., a storage section 1308, such as a hard disk; and a communication section 1309, such as a network interface card (NIC) with LAN cards, modems, etc. The communication section 1309 performs communication processing over a network such as the Internet. A drive 1310 is also connected to the input/output (I/O) interface 1305 as needed. A removable medium 1311, such as a disk, optical disk, magneto-optical disk, semiconductor memory, etc., can be mounted on the drive 1310, allowing computer programs to be read from it and installed into storage section 1308 as needed.

The present invention also provides a computer-readable storage medium, which may be included in the devices/systems described in the embodiments above or may exist separately and not be incorporated into the devices/systems. The computer-readable storage medium carries one or more programs, and when executed, these programs implement the method according to the embodiment of the present invention.

According to an embodiment of the present invention, the computer-readable storage medium may be a non-volatile computer-readable storage medium, which may include but is not limited to: portable computer disks, hard disks, random access memory (RAM), read-only memory (ROM), erasable programmable read-only memory (EPROM or Flash memory), portable compact disk read-only memory (CD-ROM), optical storage devices, magnetic storage devices, or any suitable combination of the above. In the present invention, the computer-readable storage medium can be any tangible medium that contains or stores programs that can be used by or in combination with an instruction execution system, device, or apparatus. For example, according to an embodiment of the present invention, the computer-readable storage medium may include ROM 1302 and/or RAM 1303 as described above, as well as one or more memories other than ROM 1302 and RAM 1303.

An embodiment of the present invention also includes a computer program product comprising a computer program, which contains program code for executing the method shown in the flowchart. When the computer program product is run in a computer system, the program code enables the computer system to implement the gait information processing method provided by the embodiment of the present invention.

When executed by the processor 1301, the computer program performs the functions defined in the system/device of the embodiment of the present invention. According to an embodiment of the present invention, the systems, devices, modules, units, etc., described above may be implemented by computer program modules.

In one embodiment, the computer program may reside in a tangible storage medium such as optical storage devices, magnetic storage devices, etc. In another embodiment, the computer program may be transmitted and distributed in the form of signals over a network medium and be downloaded and installed via the communication section 1309 and/or be installed from the removable medium 1311. The program code contained in the computer program may be transmitted over any suitable network medium, including but not limited to wireless, wired, etc., or any suitable combination of the above.

In such an embodiment, the computer program can be downloaded and installed from the network via the communication section 1309 and/or be installed from the removable medium 1311. When executed by the processor 1301, the computer program performs the functions defined in the system of the embodiment of the present invention. According to an embodiment of the present invention, the systems, devices, modules, units, etc., described above may be implemented by computer program modules.

According to an embodiment of the present invention, the program code for executing the computer program provided by the embodiment of the present invention can be written in any combination of one or more programming languages, specifically, it can be implemented using high-level procedural and/or object-oriented programming languages and/or assembly/machine languages. Programming languages include but are not limited to Java, C++, Python, “C” language, or similar programming languages. The program code may execute entirely on the user's computing device, partly on the user's computing device, partly on a remote computing device, or entirely on a remote computing device or server. In cases involving remote computing devices, the remote computing device may be connected to the user's computing device via any type of network, including a local area network (LAN) or a wide area network (WAN), or it may be connected to an external computing device (e.g., using an Internet Service Provider to connect via the Internet).

The flowcharts and block diagrams in the accompanying figures illustrate the possible implementation architecture, functionality, and operation of systems, methods, and computer program products according to various embodiments of the present invention. In this regard, each block in the flowchart or block diagram may represent a module, segment, or part of code, which contains one or more executable instructions for implementing the specified logical function. It should also be noted that in some alternative implementations, the functions noted in the block may occur in a different order than the one depicted in the figures. For example, two blocks shown in succession may be executed substantially in parallel, or they may sometimes be executed in the reverse order, depending on the functionality involved. It should also be noted that each block in the block diagrams or flowcharts, and combinations of blocks in the block diagrams or flowcharts, can be implemented by a dedicated hardware-based system that performs the specified functions or operations or by a combination of dedicated hardware and computer instructions.

It should be noted that unless otherwise specified that there is an execution order between different operations or there is an execution order between different operations in terms of technical implementation, the execution order of multiple operations may not be sequential, and multiple operations may also be performed simultaneously.

Those skilled in the art will understand that the features described in the various embodiments of the present invention can be combined and/or integrated in many ways, even if such combinations or integrations are not explicitly described in the present invention. In particular, without departing from the spirit and teachings of the present invention, the features described in the various embodiments of the present invention can be combined and/or integrated in many ways. All such combinations and/or integrations fall within the scope of the present invention.

The embodiments of the present invention have been described above. However, these embodiments are merely for illustrative purposes and are not intended to limit the scope of the present invention. Although the various embodiments have been described separately above, this does not imply that the measures in each embodiment cannot be used advantageously in combination. Without departing from the scope of the present invention, those skilled in the art may make various alternatives and modifications, all of which should fall within the scope of the present invention.

What is claimed is:

1-6. (canceled)

7. A method for processing gait information, applicable to wearable devices, comprising:
in response to receiving confirmation information indicating that a target subject has used a specified drug, detecting a motion state of the target subject;
when the motion state of the target subject is walking in a straight line, collecting walking information of the target subject during straight-line walking;
in response to detecting that the target subject transitions from straight-line walking to other types of walking, a number of times the walking information during straight-line walking has been collected is greater than or equal to a predefined collection threshold, and a number of daily collection cycles of the walking information during straight-line walking is greater than or equal to a predefined collection cycle threshold, processing the walking information during straight-line walking of the target subject using an objective function to obtain gait parameters; and
sending the gait parameters to a mobile communication terminal to enable the mobile communication terminal to perform anomaly detection on the gait parameters;
wherein the walking information comprises first-segment walking information, second-segment walking information, and third-segment walking information collected sequentially according to a collection time; and the method further comprises:
determining first straight-line walking status information of the target subject based on the first-segment walking information and the third-segment walking information;
in response to the first straight-line walking status information indicating that the target subject transitions from straight-line walking to turning, determining second straight-line walking status information of the target subject based on the second-segment walking information; and
determining that the target subject transitions from straight-line walking to other types of walking based on the second straight-line walking status information,
wherein the first-segment walking information comprises first walking position coordinates, and the third-segment walking information comprises second walking position coordinates; and the step of determining the first straight-line walking status information of the target subject based on the first-segment walking information and the third-segment walking information comprises:
inputting the first walking position coordinates into a linear fitting function to obtain a first fitted line, wherein the first fitted line comprises first predicted position coordinates corresponding to the first walking position coordinates, and a sum of squared errors between the first predicted position coordinates and the first walking position coordinates is minimized;
inputting the second walking position coordinates into the linear fitting function to obtain a second fitted line, wherein the second fitted line comprises second predicted position coordinates corresponding to the second walking position coordinates, and a sum of squared errors between the second predicted position coordinates and the second walking position coordinates is minimized; and
when a sum of squared errors between the first fitted line and the second fitted line satisfies a predefined error condition, determining the first straight-line walking status information of the target subject based on a first slope of the first fitted line and a second slope of the second fitted line.

8. The method according to claim 7, wherein the second-segment walking information comprises Q yaw angle values, wherein the Q yaw angle values are arranged in a walking sequence of the target subject, and Q is a positive integer greater than 1;
in response to the first straight-line walking status information indicating that the target subject transitions from straight-line walking to turning, the step of determining the second straight-line walking status information of the target subject based on the second-segment walking information comprises:
determining a difference between a qₜₕ yaw angle value and a (q+1)ₜₕ yaw angle value among the Q yaw angle values to obtain Q−1 differences, wherein q is a positive integer less than Q;
in response to a sum of the Q−1 differences being greater than or equal to a predefined difference threshold, generating the second straight-line walking status information indicating that the target subject is turning; and
in response to the sum of the Q−1 differences being less than the predefined difference threshold, generating the second straight-line walking status information indicating that the target subject is walking in the straight line.

9. The method according to claim 7, wherein the gait parameters comprise:
swing phase duration, stance phase duration, foot height, stride width, stride length, pitch angle velocity variation coefficient during foot landing phase, pitch angle velocity variation coefficient during foot push-off phase, foot height variation coefficient, stride width variation coefficient, and stride length variation coefficient.

10. A gait information processing method, applied to a mobile communication terminal, comprising:
receiving gait parameters from a wearable device, wherein the gait parameters are obtained by the wearable device in response to detecting that a target subject transitions from straight-line walking to other types of walking, a number of times the target subject's straight-line walking information has been collected is greater than or equal to a predefined collection threshold, and a number of daily collection cycles of straight-line walking information is greater than or equal to a predefined collection cycle threshold, the straight-line walking information is processed using an objective function and is collected when a motion state of the target subject is detected as walking, the motion state being detected in response to receiving confirmation information indicating that the target subject has used a specified drug; and
performing anomaly detection on the gait parameters to obtain anomaly detection results, comprising:
in response to a number of days corresponding to collected walking information being equal to a predefined day threshold, processing the gait parameters using a first gait parameter anomaly detection algorithm to obtain a first anomaly detection result corresponding to a variation pattern of the gait parameters; and
processing the gait parameters using a second gait parameter anomaly detection algorithm to obtain a second anomaly detection result corresponding to a deviation of the gait parameters;
wherein the gait parameters comprise M sets, wherein the M sets of gait parameters correspond to M medication periods of the target subject within a single day, and the M sets of gait parameters are arranged sequentially according to walking times, wherein M is a positive integer greater than 1;
wherein the step of processing the gait parameters using the first gait parameter anomaly detection algorithm to obtain the first anomaly detection result corresponding to the variation pattern of the gait parameters comprises:
calculating a first-order forward difference value of an (m−1)ₜₕ set and an mₜₕ set of gait parameters among the M sets, wherein m is a positive integer greater than 1 and less than or equal to M;
calculating a second-order forward difference value of an (m−2)ₜₕ first-order forward difference value and an (m−1)ₜₕ first-order forward difference value among M−1 first-order forward difference values;
in response to M−2 second-order forward difference values being greater than a predefined difference threshold, generating the first anomaly detection result indicating no abnormal condition in the gait parameters; and
in response to the M−2 second-order forward difference values being less than or equal to the predefined difference threshold, generating the first anomaly detection result indicating a presence of an abnormal condition in the gait parameters.

12. The gait information processing method according to claim 11, wherein the gait parameters in an mₜₕ group comprise K gait parameters corresponding to K detection cycles, and the step of processing the gait parameters using the second gait parameter anomaly detection algorithm to obtain the second anomaly detection result comprises:
determining a statistical value of the K gait parameters;
determining K differences between the statistical value and each of the K gait parameters;
in response to each of the K differences being less than or equal to a preset threshold, generating the second anomaly detection result indicating that there are no abnormal conditions in the gait parameters; and
in response to any of the K differences being greater than the preset threshold, generating the second anomaly detection result indicating a presence of abnormal conditions in the gait parameters.

13. The gait information processing method according to claim 11, further comprising:
in response to both the first anomaly detection result and the second anomaly detection result indicating no abnormalities, sending the gait parameters to a server so that the server stores the gait parameters; and
in response to at least one of the first anomaly detection result and the second anomaly detection result indicating a presence of abnormalities, sending a request to the wearable device to obtain walking information corresponding to abnormal gait parameters stored in the wearable device's cache, and sending the walking information to the server for storage.

14. The gait information processing method according to claim 12, further comprising:
in response to both the first anomaly detection result and the second anomaly detection result indicating no abnormalities, sending the gait parameters to a server so that the server stores the gait parameters; and
in response to at least one of the first anomaly detection result and the second anomaly detection result indicating a presence of abnormalities, sending a request to the wearable device to obtain walking information corresponding to abnormal gait parameters stored in the wearable device's cache, and sending the walking information to the server for storage.
